(19) United States
(12) Patent Application Publication
(10) Pub. No.: US 2017/0119958 A1
(43) Pub. Date: May 4, 2017

(54) BAG PUMP

(71) Applicant: Ahmed Abdullah Hessam, Hillerød (DK)

(72) Inventor: Ahmed Abdullah Hessam, Hillerød (DK)

(21) Appl. No.: 15/408,588

(22) Filed: Jan. 18, 2017

Related U.S. Application Data
(63) Continuation-in-part of application No. PCT/DK2015/000049, filed on Nov. 25, 2015.

(30) Foreign Application Priority Data
Nov. 26, 2014 (DK) PA 2014 00686

(51) Int. Cl. A61M 5/142

(52) U.S. Cl. CPC A61M 5/14232

(57) ABSTRACT

A bag pump for dispensing a liquid from a compressible bag to a patient is disclosed. In an embodiment the bag pump includes a box having a rear wall, an opposite front wall, two side walls and a floor between the walls. The bag box includes a propulsion spring mechanism attached at a first end thereof to one of the walls. The bag pump further includes a roller fixed to a second end of the propulsion spring mechanism The roller is adapted to squeeze the compressible bag between the roller and the floor by action of the propulsion spring mechanism.

### RELATED APPLICATIONS

This application is a continuation in part application of PCT/DK2015/000049 claiming priority from DK patent application PA201400686, both applications being hereby incorporated by reference into this disclosure with the proviso that in case of any inconsistence between the subject matter explicitly disclosed herein and the incorporated subject matter, the content of the subject matter explicitly disclosed herein prevail.

### TECHNICAL FIELD

The invention generally relates to dispensing of liquid from a compressible bag, such as an intravenous IV bag to a patient.

### BACKGROUND ART

Patients are often, during hospitalization, getting IV treatment in which they get added liquid and medicine into a vein. It is necessary to elevate the IV bag with liquid and medicine, so that the liquid can flow into the vein. For IV treatments such as these IV poles are the most commonly used tools.

IV treatments and IV poles are a significant part of both patients and the healthcare staffs everyday life in many different departments of hospitals.

The disadvantage with the IV pole occurs when nurses and paramedics have to mobilize patients. It often happens that the hospital staff has to allocate additional resources to mobilize patients.

Furthermore the IV poles occupies a lot of space in the wards and causes the staff poor working conditions due to the fact that they are either in unpleasant working positions or have to adjust the IV poles height to cause the liquid to run stronger.

Patients who are dependent of an IV pole may also increase problems with coordination among interdisciplinary staff such as nurses, occupational therapists and physiotherapists at the hospitals. Waiting time can occur, if per example a patient must retrain and at the same time complete an IV treatment.

Another frequent issue hospitals have, is that there is lack of IV poles with wheels. This means that patients are limited in their activities and will therefore need to remain in bed because of their dependence of the IV pole. It is therefore not unusual that patients are kept unnecessary long time in bed, which again can lead to unnecessary immobilization and increase the risk of bedsores.

Furthermore IV poles reduce opportunities for children to participate in games and other activities due to dependence of IV poles.

Initiation of births frequently takes place while a pregnant woman is given IV treatment. In this situation mobilization of the pregnant would be an absolute advantage, because she then would be able to move around freely until the birth has really started.

In acute accidents or treatment of patients outside hospitals and ambulance, it is often required that a person acts as an IV pole.

There have been many attempts to provide a bag pump which does not require a pole.

Smiths Medical, Care Fusion, Baxter and Micrel produces electronic infusion pumps that can make patients independent of the IV pole. Such electronic infusion pumps are however relative expensive.

U.S. Pat. No. 4,850,971 describes an infusion pump utilizing a linear roller driven by a one or more constant force springs in combination with changeable flow regulating needles to provide a constant flow, gravity independent device. The constant spring springs comprises a coil type that is mounted to a storage drum and an end of the coil springs are attached to a plate such that when the drum is pulled away from the plate the spring is unrolled. In use the bag is rolled onto drum between windings of the coil spring.

### DISCLOSURE OF INVENTION

An object of the present invention is to provide a bag pump which is independent of electrical power, and which is also reliable.

In an embodiment it is an object to provide a mechanical powered pump suitable for fully or partly emptying a compressible bas, such as an IV bag.

In an embodiment it is an object to provide a bag pump which is mobile and which is operating essentially gravity independent.

In an embodiment it is an object to provide a bag pump which enables a patient to move freely around e.g. to train while being under intravenous treatment with the pump.

In an embodiment it is an object to provide a bag pump suitable for treatment of patients outside hospitals e.g. in private homes and/or in ambulance.

In an embodiment it is an object to provide a bag pump for reliable dispensing of liquid from a collapsible bag, such as an IV bag, which bag pump is relatively inexpensive and is simple to use.

In an embodiment it is an object to provide a bag pump system comprising a bag pump and a compressible bag, which bag pump system is simple and safe to use and with a high reliability and low risk of damaging the bag during dispensing of the liquid from the bag.

In an embodiment it is an object to provide a method of dispensing liquid from a compressible bag to a patient, which method is highly reliably and with low risk of damaging the bag during dispensing of the liquid from the bag.

This and other objects have been solved by the invention or embodiments thereof as defined in the claims or as described herein below.

It has been found that the invention or embodiments thereof have a number of additional advantages, which will be clear to the skilled person from the following description.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

Throughout the description or claims, the singular encompasses the plural unless otherwise specified or required by the context.

All features of the inventions and embodiments of the invention as described herein including ranges and preferred ranges may be combined in various ways within the scope of the invention, unless there are specific reasons not to combine such features.

In an embodiment the bag pump comprises a box with a rear wall, an opposite front wall and a floor between the rear wall and the front wall. The floor may advantageously interconnect the rear wall and the front wall.

In an embodiment the bag pump comprises a box with the rear wall, the opposite front wall, two side walls and the floor between the walls. The floor is advantageously essentially flat.

The bag pump comprises propulsion spring mechanism having a first and a second end. The first end of the propulsion spring mechanism is fixed to at least one of the walls, such as to the rear wall or to the front wall front wall or to the two side walls depending on the type of propulsion spring mechanism. The second end of the propulsion spring mechanism is fixed to a roller which is adapted to squeeze a compressible bag between the roller and the floor by action of the propulsion spring mechanism for thereby dispensing a liquid from the compressible bag e.g. for dispensing the liquid to a patient. It has been found that the bag pump is highly reliable and the risk of damaging the bag during dispensing is very low. Advantageously the contact between the roller and the bag is a firm contact essentially without frictional force but a simple rolling of the roller over the bag while the roller is pressing the bag towards the floor, preferably without the bag being removed from the floor. Thus the bag pump may ensure a simple and safe dispensing of the liquid.

The roller is advantageously adapted for squeezing the compressible bag between the roller and the floor for thereby dispensing the liquid from the bas while the bag remain in full face-to-face contact with the floor.

To ensure that the bag is not rolled onto the roller the bag advantageously comprises a bag fastener for fastening the bag to the bag box. The bag fastener may for example be or comprise a hook, a button, a buckle and/or Velcro. The bag fastener is advantageously positioned at the floor closer to the rear wall than to the front wall, such as at or near the rear wall.

In an embodiment the type of propulsion spring mechanism is a compression spring mechanism where the propulsion spring mechanism is compressed from its resting position to a loaded condition. In this embodiment the first end of the propulsion spring mechanism is advantageously fixed to the rear wall.

In an embodiment the type of propulsion spring mechanism is a tensile spring mechanism where the propulsion spring mechanism is stretched from its resting position to a loaded condition. In this embodiment the first end of the propulsion spring mechanism is advantageously fixed to the front wall.

In an embodiment the propulsion spring mechanism is adapted to be loaded by pushing the roller towards to rear wall to expose a bag receiving section of the box between the front wall and the roller. The floor in the bag receiving section is advantageously uncovered prior to application of the bag, i.e. the bag may be applied directly onto the floor in the bag receiving section essentially without any elements between the floor and the bag and in particular without any spring element(s) or part(s) thereof between floor and the bag in the bag receiving section.

In an embodiment the propulsion spring mechanism is fixed to the rear wall of the bag and the propulsion spring mechanism is a compression spring mechanism, such as a telescopic conveyance mechanism.

The telescopic conveyance mechanism may for example comprise two or more elements that are telescopically connected. Optionally the telescopically interconnected elements comprises a blocking or locking mechanism adapted for holding the elements engaged to each other. In another or in the same embodiment the telescopically interconnected elements are held in engaged telescopic configuration by the box.

In an embodiment the propulsion spring mechanism comprises at least one spring arranged to provide the propulsion. The spring may be a compression spring or a tensile spring.

The propulsion spring mechanism may comprise two or more springs of same or different type. In an embodiment the telescopic conveyance mechanism comprises at least one spring for each telescopically interconnected element arranged to provide the propulsion.

In order to reduce the risk or fully ensure that the bag is not entangled in the spring or springs of the propulsion spring mechanism the propulsion spring mechanism advantageously comprises an encasing which fully encases the spring(s) of the propulsion spring mechanism. The encasing may for example be fully or partly provided by the telescopically interconnected elements.

In an embodiment where the propulsion spring mechanism comprises an encasing, the roller is fixed to the encasing, preferably such that it can freely roll independently of the propulsion spring mechanism. The roll may for example comprise an axle and a connecting arrangement fixing (e.g. rigidly fixing) the axle to the encasing of the propulsion spring mechanism.

By ensuring that the spring(s) of the propulsion spring mechanism is physically separated from the roller as the roller rolls over the bag, the risk of damaging the bag during the dispensing may be reduced or even fully avoided.

The bag pump advantageously has an orifice in the front wall for dispensing liquid from the compressible bag. For example a dispensing tip and/or a dispensing tube may be passed through the orifice and e.g. connected to a needle for dispensing the liquid.

The orifice may advantageously form a collar suitably for holding a dispensing tube of the bag in a fixed position. While simultaneously fastening the bag to the floor e.g. at a rear bag end opposite the dispensing tube, the roller may be driven to roll over the bag to dispensing the liquid from the bag without risking that the bag is rolled onto the roller.

To ensure an even and continuous dispensing it is generally desired that the floor at least in the bag receiving section is essentially flat i.e. such than when the roller is rolling over the bag while simultaneously pressing the bag towards the floor the liquid is dispensed in a evenly and continuous stream.

The invention also comprises a bag pump system comprising a bag pump and a compressible bag.

In an embodiment the bag bump is as described above.

In an embodiment the bag pump comprises a box having a rear wall, an opposite front wall, two side walls and a floor between the walls. The bag pump comprises a propulsion spring mechanism attached at a first end thereof to one of the rear and front walls and a roller fixed to a second end of the propulsion spring mechanism. The propulsion spring mechanism is loadable by pushing the roller towards the rear wall to expose a bag receiving section of the box between the front wall and the roller for receiving and preferably fastening the compressible bag in direct contact with the floor in the bag receiving section.

The bag is advantageously adapted to fit into the bag receiving section to be applied directly in interfacial contact with the floor. The floor is advantageously essentially flat and the bag is applied in full contact with the floor without any intermediate elements.

Advantageously the roller is adapted to roll over and squeeze the compressible bag towards the floor by action of the propulsion spring mechanism without the bag being rolled onto the roller.

In an embodiment a bag pump system comprises the bag pump and a compressible bag. The bag pump may be as described above. The propulsion spring is loadable by pushing the roller towards the rear wall to expose the bag receiving section for receiving and fastening the compressible bag in direct contact with the floor in the bag receiving section. Advantageously the roller is adapted to roll over and squeeze the compressible bag towards the flat floor by action of the propulsion spring mechanism while the bag remain fastened to the box in said direct contact with the floor.

The invention also comprises a method of dispensing liquid from a compressible bag to a patient. In an embodiment the method comprises providing a bag pump with a propulsion spring mechanism and a roller, loading the compressible bag into the bag pump and rolling the roller over the bag by the propulsion spring mechanism to thereby dispense the liquid.

In an embodiment the bag pump is as described above.

In an embodiment the bag pump comprises box with a rear wall, an opposite front wall and a floor between the rear wall and the front wall and the propulsion spring mechanism attached to the roller e.g. as described above.

The method preferably comprises loading the propulsion spring mechanism by pushing the roller towards to rear wall to expose a bag receiving section of the box between the front wall and the roller, positioning the compressible bag directly onto the floor in the bag receiving section and releasing the propulsion spring mechanism to force the roller over the bag to thereby dispensing the liquid.

In an embodiment the method comprises manually loading the propulsion spring mechanism.

Advantageously the box comprises a bag fastener and the positioning of the bag in the bag receiving section comprises fastening the bag to the box by said bag fastener, e.g. by hanging the bag onto a hook. Thereby it may be ensured that the roller may be rolled over the bag for dispensing the liquid in the bag without the bag being rolled onto the roller. Thus the fastener ensures that the bag may remain in full contact with the floor during dispensing.

Advantageously the method comprises squeezing the compressible bag between the roller and the floor by action of the propulsion spring mechanism and preferably by the propulsion spring mechanism pushing the roller to roll over the bag while simultaneously pressing the bag towards the floor.

Preferably the compressible bag remain in full contact with the floor in the bag receiving section during the dispensing, preferably such that the compressible bag is not rolled onto the roller.

### BRIEF DESCRIPTION OF EXAMPLES OF EMBODIMENTS AND ELEMENTS OF THE INVENTION

The above and/or additional objects, features and advantages of the present invention will be further elucidated by the following illustrative and non-limiting description of embodiments of the present invention, with reference to the appended drawings.

The figures are schematic and are not drawn to scale and may be simplified for clarity. Throughout, the same reference numerals are used for identical or corresponding parts.

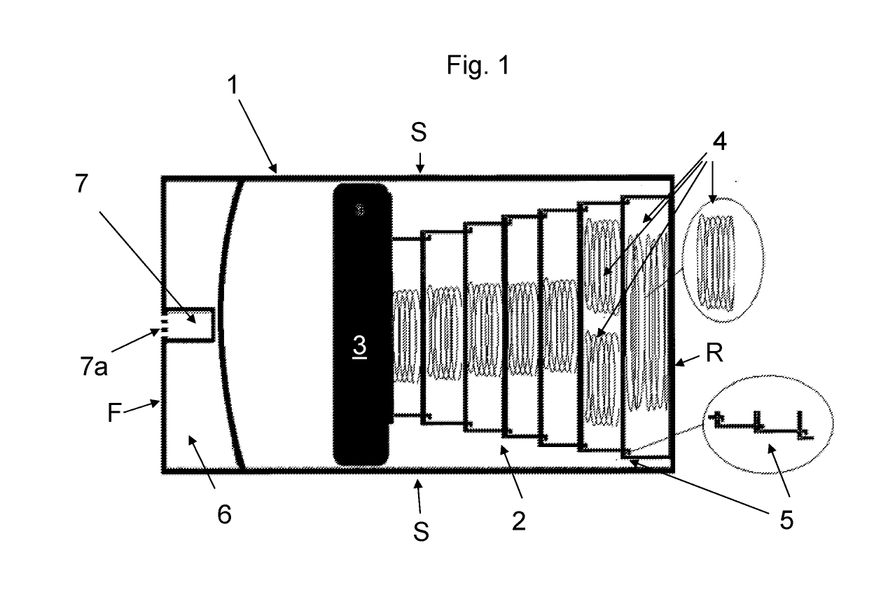

FIG. 1 is a schematic top view of an embodiment of a bag pump of the invention.

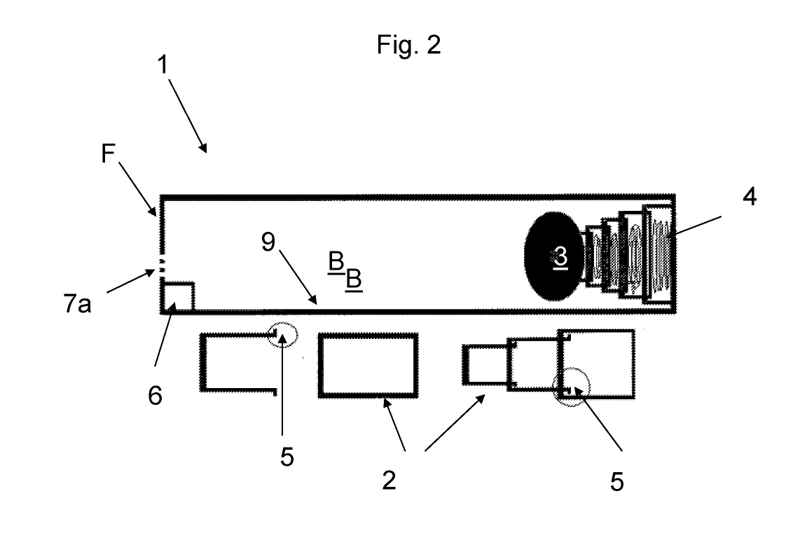

FIG. 2 is a schematic side view of the embodiment shown in FIG. 1 and elements thereof.

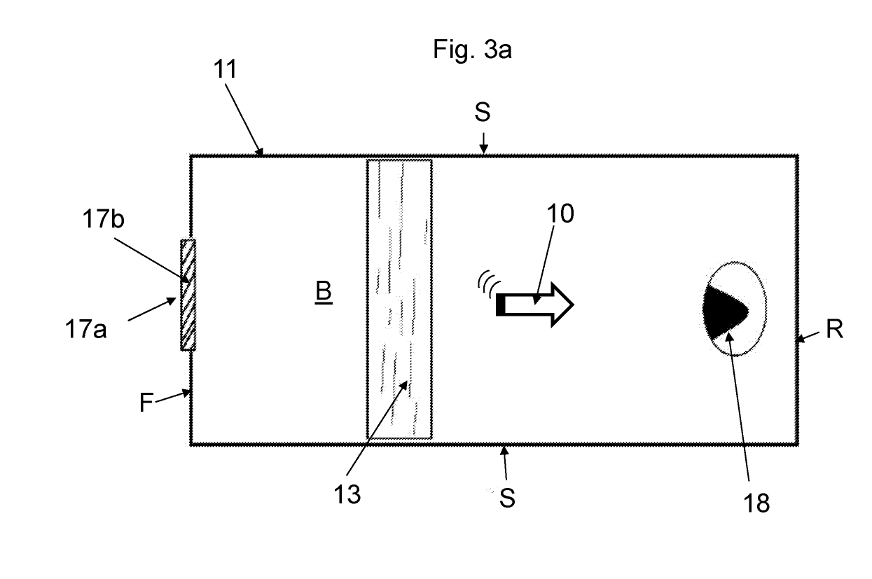

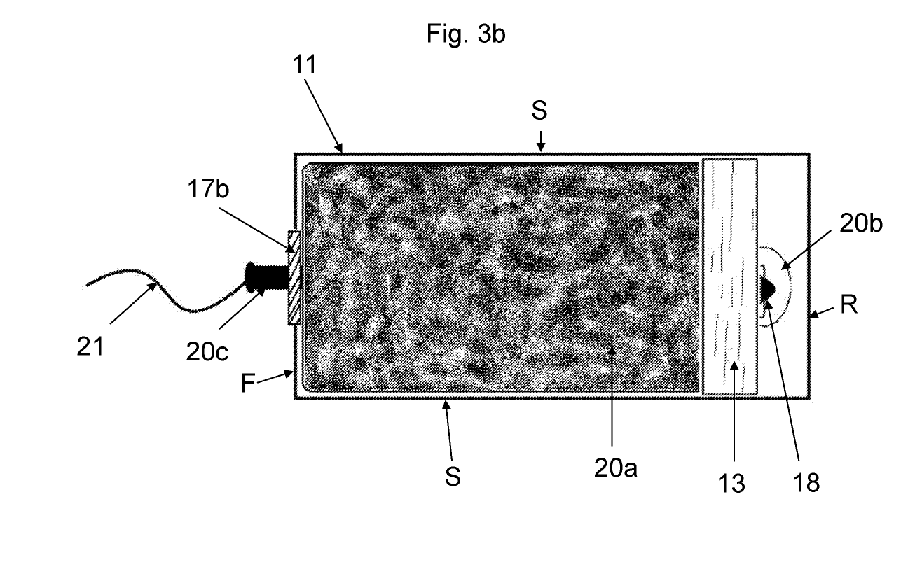

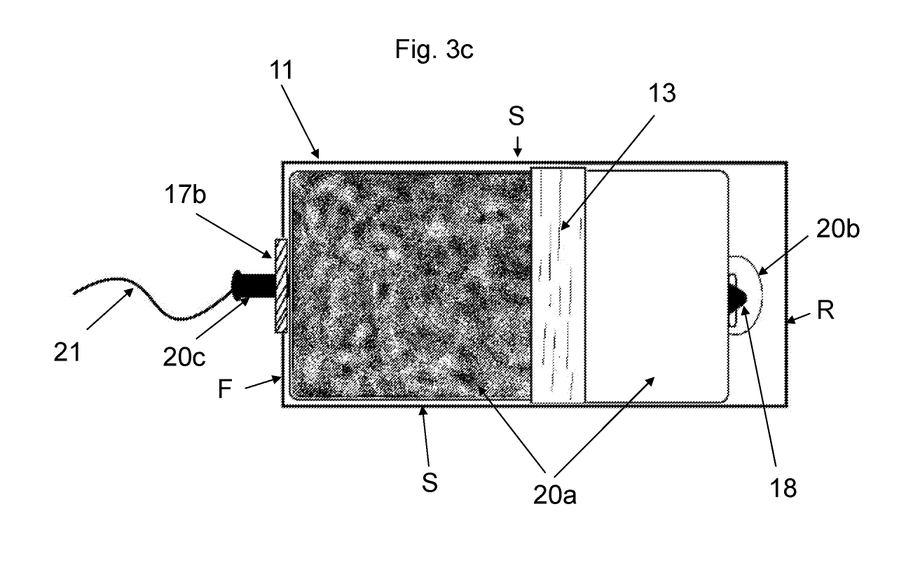

FIG. 3a is a schematic top view of an embodiment of a bag pump of the invention wherein the propulsion spring arrangement is not shown.

FIG. 3b is a schematic top view of the bag pump of FIG. 3a wherein an infusion bag has been arranged.

FIG. 3c is a schematic top view of the bag pump with bag of FIG. 3b wherein the infusion has started.

FIGS. 1 and 2 show an example of an embodiment of a bag pump of the invention, in this embodiment an infusion bag bump, such as an IV bag pump. Instead of elevating an IV bag containing liquid/medicine, the IV bag is placed in a small box 1, which can for example be placed in a small backpack on the patient. The IV bag pump comprises a box 1, in which there can be placed a not shown IV bag. In the box there is a roller 3, which is adjusted to press the liquid out of an IV bag, which is placed in the box. After pulling back the roller 3 in the box 1 an IV bag is inserted into the IV bag pump. In the shown embodiment the roller 3 has an oval cross sectional profile. In a variation thereof the roller has a round cross sectional profile. There is also attached a telescopic 2 conveyance mechanism on the roller 3 and its function is to drive and guide the roller 3 to roll over the IV bag, and in that way develop the necessary pressure to empty the IV bag. This occurs as the patient absorbs the liquid from the IV bag.

The telescopic 2 conveyance mechanism comprises a number of elements that are telescopically interconnected 2, one inside the other. Each element has a blocking / locking mechanism 5 which ensures that the components remain engaged. The other end of the conveyance mechanism is attached to the box's 1 rear wall. The conveyance mechanism furthermore comprises a spring (4), which is arranged to provide propulsion, so that the telescopic 2 arranged elements are forced toward their outstretched form.

This allows the roller 3 only to get momentum as the patient absorbs the liquid from the IV bag. The telescopic 2 elements ensure that the spring 4 is held in place.

The IV bag pump is mechanical powered. It comprises a box (1), wherein an IV bag is placed. The IV bag is hereafter emptied by one or more springs 4, which forces a roller 3 over the IV bag. The propulsion-spring 4 is positioned within a telescoping 2 mechanism that forces the roller 3 over the IV bag, which then is emptied in a reliable way.

The box 1 comprises a front wall F, a rear wall R and two side walls S. Adjacent to the front wall F, the box 1 has a support shelf 6 with a collar shaped recess 7 to support a bag and a bag tube. An orifice 7a in the front wall F is arranged such that the bag tube can protrude out of the box 1 and at the same time the bag will be hold in position at its dispensing tube.

When the propulsion spring arrangement in the form of the telescoping 2 mechanism with the springs 4 is loaded by pushing the roller towards to rear wall R as shown in FIG. 2, the bag receiving section B of the box between the front wall F and the roller 3 is exposed. The floor 9 in the bag receiving section B is a flat floor 9. And after having positioning a bag directly onto the floor 9 in the bag receiving section B and with its dispensing tube through the orifice 7a the propulsion spring arrangement 2 may be released to force the roller 3 to roll over the bag to thereby dispense the liquid.

The bag pump shown in FIGS. 3a, 3b, 3c comprises a box 11 with a rear wall R, an opposite front wall F and two side walls S. The bag pump comprises a roller 13 fixed to a not shown said propulsion spring mechanism such that when the roller 13 is moved to a loaded position near the rear wall R a bag receiving section B with a flat floor is exposed.

The box 1 has an orifice 17a at its front wall and a locking frame 17b for locking a dispenser tube in position. The box 1 further comprises a bag fastener 18, which in the shown embodiment is a hinge 18.

In FIG. 3a the roller 13 is moved towards the rear wall as indicated with the arrow 10 to thereby load the propulsion spring mechanism and expose the bag receiving section B.

In FIG. 3b an infusion bag 20a, with a hanger loop 20b and a dispenser tube 20c is positioned in the bag receiving section B. The hanger loop 20b is hanged onto the hinge 18 and the dispenser tube 20c is passed through the orifice and locked be the locking frame 17b. Thereby the infusion bag 20a is secured against the flat floor in the bag receiving section B. A transfer tube 21 is connected to the dispenser tube 20c for guiding dispensed fluid to a patent.

In FIG. 3c the propulsion spring mechanism has been released and the propulsion spring mechanism drives the roller 13 to roll over the infusion bag 20a to dispense the liquid via the dispenser tube 20c to the patient. The fastening of the infusion bag 20a by the hanger loop 20b to the bag hanger 18 ensures that the infusion bag remains in close contact with the floor of the bag receiving section B and that the bag is not rolled onto the roller 13.

Further scope of applicability of the present invention will become apparent from the description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

1. A bag pump for dispensing a liquid from a compressible bag to a patient, the bag pump comprising a box having a rear wall, an opposite front wall, two side walls and a floor between the walls and comprising a propulsion spring mechanism attached at a first end thereof to one of said walls, wherein said bag pump comprises a roller fixed to a second end of said propulsion spring mechanism, wherein when the roller is adapted to squeeze said compressible bag between said roller and said floor by action of said propulsion spring mechanism.

2. The bag pump of claim 1, wherein said propulsion spring mechanism is fixed to said rear wall of said bag and wherein said propulsion spring mechanism being loadable by pushing the roller towards to rear wall to expose a bag receiving section of the box between the front wall and the roller, wherein the floor in the bag receiving section is uncovered prior to application of said bag.

3. The bag pump of claim 1, wherein said propulsion spring mechanism is a telescopic conveyance mechanism.

4. The bag pump of claim 3, wherein said telescopic conveyance mechanism comprises a number of elements that are telescopically interconnected.

5. The bag pump of claim 4, wherein said telescopically interconnected elements comprises a blocking or locking mechanism adapted for holding the elements engaged to each other.

6. The bag pump of claim 1, wherein said propulsion spring mechanism comprises at least one spring arranged to provide the propulsion.

7. The bag pump of claim 4, wherein said propulsion spring mechanism comprises at least one spring for each telescopically interconnected element arranged to provide the propulsion.

8. The bag pump of claim 6, wherein said propulsion spring mechanism comprises an encasing which fully encases said at least one spring.

9. The bag pump of claim 6, wherein said at least one spring is physically separated from said roller.

10. The bag pump of claim 1, wherein said wherein said roller is fixed to an encasing of said propulsion spring mechanism.

11. The bag pump of claim 1, wherein said bag pump having an orifice in said front wall for dispensing liquid from said compressible bag.

12. The bag pump of claim 2, wherein at least said floor in the bag receiving section is essentially flat.

13. The bag pump of claim 1, wherein the box comprises a bag fastener for fastening the bag to the bag box.

14. A method of dispensing liquid from a compressible bag to a patient, the method comprising providing a bag pump comprising a box having a rear wall, an opposite front wall, two side walls and a floor between the walls and a propulsion spring mechanism attached to a roller, the method comprising loading the propulsion spring mechanism by pushing the roller towards to rear wall to expose a bag receiving section of the box between the front wall and the roller, positioning the compressible bag directly onto the floor in the bag receiving section and releasing the propulsion spring mechanism to force the roller to roll over the bag to thereby dispensing the liquid.

15. The method of claim 13, wherein the box comprises a bag fastener and the positioning of the bag in the bag receiving section comprises fastening said bag to the box by said bag fastener.

16. The method of claim 14, wherein the method comprises manually loading said propulsion spring mechanism.

17. The method of claim 14, wherein the bag pump having an orifice in the front wall and the method comprises dispensing said liquid via said orifice.

18. The method of claim 14, wherein the method comprises squeeze said compressible bag between said roller and said floor by action of said propulsion spring mechanism.

19. The method of claim 14, wherein compressible bag remain in full contact with the floor in the bag receiving section during the dispensing.

20. The method of claim 14, wherein the roller rolls over the bag for dispensing the liquid in the bag without the bag being t rolled onto the roller.

21. A bag pump system comprising a bag pump and a compressible bag, wherein said the bag pump comprises a box having a rear wall, an opposite front wall, two side walls and a floor between the walls and comprising a propulsion spring mechanism attached at a first end thereof to one of said walls, wherein said bag pump comprises a roller fixed to a second end of said propulsion spring mechanism and said propulsion spring mechanism being loadable by pushing the roller towards the rear wall to expose a bag receiving section of the box between the front wall and the roller for receiving and fastening said compressible bag in direct contact with the floor in the bag receiving section.

22. The bag pump system of claim 21, wherein when the roller is adapted to roll over and squeeze said compressible bag towards said floor by action of said propulsion spring mechanism while said bag remain fastened to said box in said direct contact with the floor.
